(19) United States
(12) Patent Application Publication
(10) Pub. No.: US 2014/0182018 A1
(43) Pub. Date: Jun. 26, 2014

(54) Combinations of Cry1Ab and Cry1Fa as an insect resistance management tool

(71) Applicant: Agrigenetics, Inc., Indianapolis, IN (US)

(72) Inventors: Bruce A. Lang, Maxwell, IA (US); James W. Bing, Zionsville, IN (US); Jonathan M. Babcock, Carmel, IN (US); Thomas Meade, Zionsville, IN (US); Nicholas P. Storer, Kensington, MD (US); Blair David Siegfried, Lincoln, NE (US); Elisen Jose Guedes Pereira, Lincoln, NE (US)

(73) Assignee: Agrigenetics, Inc., Indianapolis, IN (US)

(21) Appl. No.: 14/082,828

(22) Filed: Nov. 18, 2013

Related U.S. Application Data
(63) Continuation of application No. 12/156,515, filed on Jun. 2, 2008, now abandoned.
Application No. 12/156,515 is a continuation of application No. 11/072,812, filed on Mar. 4, 2005, now abandoned.
(60) Provisional application No. 60/550,645, filed on Mar. 5, 2004.

(51) Int. Cl. A01N 63/02; C12N 15/82

(52) U.S. Cl. CPC A01N 63/02; C12N 15/8286
USPC 800/302; 424/93.21; 514/4.5

(57) ABSTRACT

Compositions for controlling lepidopteran insects use Cry1Fa and Cry1Ab core toxin containing proteins in combination to delay or prevent development of resistance.

### CROSS REFERENCE

This application is a continuation of U.S. application Ser. No. 12/156,515, filed Jun. 2, 2008, which claims the priority of U.S. application Ser. No. 11/072,812, filed Mar. 4, 2005, now abandoned, which claims the benefit of U.S. provisional application Ser. No. 60/550,645, filed Mar. 5, 2004. The entire disclosures of which are expressly incorporated herein by reference.

### BACKGROUND OF THE INVENTION

Billions of dollars are spent each year to control insect pests and additional billions are lost to the damage they inflict. Synthetic organic chemical insecticides have been the primary tools used to control insect pests but biological insecticides, such as the insecticidal proteins derived from Bacillus thuringiensis (Bt), have played an important role in some areas. The ability to produce insect resistant plants through transformation with Bt insecticidal protein genes has revolutionized modern agriculture and heightened the importance and value of insecticidal proteins and their genes.

Several Bt proteins have been used to create the insect-resistant transgenic plants that have been successfully registered and commercialized to date. These include Cry1Ab, Cry1Ac, Cry1F and Cry3Bb in corn, Cry1Ac and Cry2Ab in cotton, and Cry3A in potato. The commercial products expressing these proteins express a single protein except in cases where the combined insecticidal spectrum of 2 proteins is desired (e.g., Cry1Ab and Cry3Bb in corn combined to provide resistance to lepidopteran pests and rootworm, respectively) or where the independent action of the proteins makes them useful as a tool for delaying the development of resistance in susceptible insect populations (e.g., Cry1Ac and Cry2Ab in cotton combined to provide resistance management for tobacco budworm).

Some of the qualities of insect-resistant transgenic plants that have led to rapid and widespread adoption of this technology also give rise to the concern that pest populations will develop resistance to the insecticidal proteins produced by these plants. Several strategies have been suggested for preserving the utility of Bt-based insect resistance traits which include deploying proteins at a high dose in combination with a refuge, and alternation with, or co-deployment of, different toxins (McGaughey et al. (1998), “B.t. Resistance Management,” Nature Biotechnol. 16:144-146).

The proteins selected for use in an IRM stack need to exert their insecticidal effect independently so that resistance developed to one protein does not confer resistance to the second protein (i.e., there is not cross resistance to the proteins). A robust assessment of cross-resistance is typically made using populations of a pest species normally sensitive to the insecticidal protein that have been selected for resistance to the insecticidal proteins. If, for example, a pest population selected for resistance to “Protein A” is sensitive to “Protein B,” we would conclude that there is not cross resistance and that a combination of Protein A and Protein B would be effective in delaying resistance to Protein A alone.

In the absence of resistant insect populations, assessments can be made based on other characteristics presumed to be related to mechanism of action and cross-resistance potential. The utility of receptor-mediated binding in identifying insecticidal proteins likely to not exhibit cross resistance has been suggested (van Mellaert et al. 1999). The key predictor of lack of cross resistance inherent in this approach is that the insecticidal proteins do not compete for receptors in a sensitive insect species.

Cry1Ab and Cry1Fa are insecticidal proteins currently used in transgenic corn to protect plants from a variety of insect pests. A key pest of corn that these proteins provide protection from is the European corn borer, Ostrinia nubilalis (Hübner). The ability to conduct receptor binding studies using Cry1Fa is limited because the technique available for labeling Cry1Fa inactivates the protein. The limited information regarding competitive binding between Cry1Ab and Cry1F in O. nubilalis indicates some competition between these 2 proteins (i.e., cross resistance) but is inadequate for the authors to make a firm conclusion regarding cross resistance potential between these 2 insecticidal proteins (Hua et al., 2001).

### BRIEF SUMMARY OF THE INVENTION

The subject invention relates to the surprising discovery that that a European corn borer population selected for resistance to Cry1Fa is not resistant to Cry1Ab. Furthermore, larvae from this Cry1F-resistant European corn borer population develop on transgenic corn plants expressing Cry1Fa but fail to develop on corn plants expressing Cry1Ab. As one skilled in the art will recognize with the benefit of this disclosure, plants expressing these 2 insecticidal proteins, or insecticidal portions thereof, will be useful in delaying or preventing the development of resistance to either of these insecticidal proteins alone.

The present invention provides

compositions for controlling lepidopteran pests comprising cells that express a Cry1F chimeric core toxin-containing protein and a Cry1Ab chimeric core toxin-containing protein;

a host transformed to express both a Cry1F core toxin-containing protein and a Cry1Ab core toxin containing protein, wherein said host is a microorganism or a plant cell;

a method of controlling lepidopteran pests comprising contacting said pests or the environment of said pests with an effective amount of a composition which produces a Cry1F chimeric core toxin-containing protein and a cell expressing a Cry1Ab chimeric core toxin-containing protein;

a maize plant comprising DNA encoding a Cry1Ab chimeric core toxin-containing protein and DNA encoding a Cry 1F core toxin-containing protein, and seed of such a plant;

a maize plant wherein DNA encoding a Cry1Ab chimeric core toxin-containing protein and DNA encoding a Cry 1F core toxin-containing protein have been introgressed into said maize plant, and seed of such a plant.

### BRIEF DESCRIPTION OF THE FIGURES

FIG. 1 is a graph showing mean (+SD) feeding damage on corn genotypes by neonates from non-selected and Cry1F-selected O. nubilalis populations.

FIG. 2 is a graph showing concentration-response curves for the susceptible population (open circles), the Cry1Fa-selected population (solid circles) and reciprocal crosses between populations (open squares and open triangles) to Cry1Fa.

### DETAILED DESCRIPTION OF THE INVENTION

SEQ ID NO:3 of U.S. Pat. No. 6,114,608 describes a synthetic Cry1Ab gene suitable for use in carrying out the present invention.

U.S. Pat. No. 6,172,285 describes an inbred corn line with the MON810 (Cry1Ab) trait that is suitable for use in carrying out the present invention.

U.S. Pat. No. 6,657,109, U.S. Pat. No. 6,646,187, U.S. Pat. No. 6,353,259, U.S. Pat. No. 6,316,701, U.S. Pat. No. 6,169,233, U.S. Pat. No. 6,166,304, U.S. Pat. No. 6,140,563, and U.S. Pat. No. 6,072,110 describe and claim inbred corn lines with the Bt11 or Event 176 (Cry1Ab) traits, suitable for use in carrying out the present invention.

U.S. Pat. No. 5,188,960 and U.S. Pat. No. 5,827,514 describe Cry1F core toxin containing proteins suitable for use in carrying out the present invention. U.S. Pat. No. 6,218,188 describes plant-optimized DNA sequences encoding Cry1F core toxin-containing proteins that are suitable for use in the present invention.

Combinations of the toxins described in the invention can be used to control lepidopteran pests. Adult lepidopterans, i.e., butterflies and moths, primarily feed on flower nectar and are a significant effector of pollination. The larvae, i.e., caterpillars, nearly all feed on plants, and many are serious pests. Caterpillars feed on or inside foliage or on the roots or stem of a plant, depriving the plant of nutrients and often destroying the plant's physical support structure. Additionally, caterpillars feed on fruit, fabrics, and stored grains and flours, ruining these products for sale or severely diminishing their value. As used herein, reference to lepidopteran pests refers to various life stages of the pest, including larval stages.

The chimeric toxins of the subject invention comprise a full core N-terminal toxin portion of a B.t. toxin and, at some point past the end of the toxin portion, the protein has a transition to a heterologous protoxin sequence. The N-terminal toxin portion of a B.t. toxin is referred to herein as the “core” toxin. The transition to the heterologous protoxin segment can occur at approximately the toxin/protoxin junction or, in the alternative, a portion of the native protoxin (extending past the toxin portion) can be retained with the transition to the heterologous protoxin occurring downstream. As an example, one chimeric toxin of the subject invention has the full toxin portion of cryIF (amino acids 1-601) and a heterologous protoxin (amino acids 602 to the C-terminus). In a preferred embodiment, the heterologous portion of the protoxin is derived from a cryIA(b) or 436 toxin.

A person skilled in this art will appreciate that B.t. toxins, even within a certain class such as cryIF, will vary to some extent in length and the precise location of the transition from toxin portion to protoxin portion. Typically, the cryIA(b) and cryIF toxins are about 1150 to about 1200 amino acids in length. The transition from toxin portion to protoxin portion will typically occur at between about 50% to about 60% of the full length toxin. The chimeric toxin of the subject invention will include the full expanse of this core N-terminal toxin portion. Thus, the chimeric toxin will comprise at least about 50% of the full length cryIF B.t. toxin. This will typically be at least about 590 amino acids. With regard to the protoxin portion, the full expanse of the cryIA(b) protoxin portion extends from the end of the toxin portion to the C-terminus of the molecule. It is the last about 100 to 150 amino acids of this portion which are most critical to include in the chimeric toxin of the subject invention.

Genes and toxins. The genes and toxins useful according to the subject invention include not only the full length sequences disclosed but also fragments of these sequences, variants, mutants, and fusion proteins which retain the characteristic pesticidal activity of the toxins specifically exemplified herein. As used herein, the terms “variants” or “variations” of genes refer to nucleotide sequences which encode the same toxins or which encode equivalent toxins having pesticidal activity. As used herein, the term “equivalent toxins” refers to toxins having the same or essentially the same biological activity against the target pests as the claimed toxins.

It should be apparent to a person skilled in this art that genes encoding active toxins can be identified and obtained through several means. The specific genes or gene portions exemplified herein may be obtained from the isolates deposited at a culture depository as described above. These genes, or portions or variants thereof, may also be constructed synthetically, for example, by use of a gene synthesizer. Variations of genes may be readily constructed using standard techniques for making point mutations. Also, fragments of these genes can be made using commercially available exonucleases or endonucleases according to standard procedures. For example, enzymes such as Bal31 or site-directed mutagenesis can be used to systematically cut off nucleotides from the ends of these genes. Also, genes which encode active fragments may be obtained using a variety of restriction enzymes. Proteases may be used to directly obtain active fragments of these toxins.

Fragments and equivalents which retain the pesticidal activity of the exemplified toxins would be within the scope of the subject invention. Also, because of the redundancy of the genetic code, a variety of different DNA sequences can encode the amino acid sequences disclosed herein. It is well within the skill of a person trained in the art to create these alternative DNA sequences encoding the same, or essentially the same, toxins. These variant DNA sequences are within the scope of the subject invention. As used herein, reference to “essentially the same” sequence refers to sequences which have amino acid substitutions, deletions, additions, or insertions which do not materially affect pesticidal activity. Fragments retaining pesticidal activity are also included in this definition.

A further method for identifying the toxins and gene portions useful according to the subject invention is through the use of oligonucleotide probes. These probes are detectable nucleotide sequences. These sequences may be detectable by virtue of an appropriate label or may be made inherently fluorescent as described in International Application No. WO93/16094. As is well known in the art, if the probe molecule and nucleic acid sample hybridize by forming a strong bond between the two molecules, it can be reasonably assumed that the probe and sample have substantial homology. Preferably, hybridization is conducted under stringent conditions by techniques well-known in the art, as described, for example, in Keller, G. H., M. M. Manak (1987) DNA Probes, Stockton Press, New York, N.Y., pp. 169-170. Detection of the probe provides a means for determining in a known manner whether hybridization has occurred. Such a probe analysis provides a rapid method for identifying toxin-encoding genes of the subject invention. The nucleotide segments which are used as probes according to the invention can be synthesized using DNA synthesizer and standard procedures. These nucleotide sequences can also be used as PCR primers to amplify genes of the subject invention.

Certain toxins of the subject invention have been specifically exemplified herein. Since these toxins are merely exemplary of the toxins of the subject invention, it should be readily apparent that the subject invention comprises variant or equivalent toxins (and nucleotide sequences coding for equivalent toxins) having the same or similar pesticidal activity of the exemplified toxin. Equivalent toxins will have amino acid homology with an exemplified toxin. This amino acid homology will typically be greater than 75%, preferably be greater than 90%, and most preferably be greater than 95%. The amino acid homology will be highest in critical regions of the toxin which account for biological activity or are involved in the determination of three-dimensional configuration which ultimately is responsible for the biological activity. In this regard, certain amino acid substitutions are acceptable and can be expected if these substitutions are in regions which are not critical to activity or are conservative amino acid substitutions which do not affect the three-dimensional configuration of the molecule. For example, amino acids may be placed in the following classes: non-polar, uncharged polar, basic, and acidic. Conservative substitutions whereby an amino acid of one class is replaced with another amino acid of the same type fall within the scope of the subject invention so long as the substitution does not materially alter the biological activity of the compound. The following list provides a listing of examples of amino acids belonging to each class.

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

In some instances, non-conservative substitutions can also be made. The critical factor is that these substitutions must not significantly detract from the biological activity of the toxin.

Recombinant hosts. The genes encoding the toxins of the subject invention can be introduced into a wide variety of microbial or plant hosts. Expression of the toxin gene results, directly or indirectly, in the intracellular production and maintenance of the pesticide. Conjugal transfer and recombinant transfer can be used to create a B.t. strain that expresses both toxins of the subject invention. Other host organisms may also be transformed with one or both of the toxin genes then used to accomplish the synergistic effect. With suitable microbial hosts, e.g., Pseudomonas, the microbes can be applied to the situs of the pest, where they will proliferate and be ingested. The result is control of the pest. Alternatively, the microbe hosting the toxin gene can be treated under conditions that prolong the activity of the toxin and stabilize the cell. The treated cell, which retains the toxic activity, then can be applied to the environment of the target pest.

Where the B.t. toxin gene is introduced via a suitable vector into a microbial host, and said host is applied to the environment in a living state, it is essential that certain host microbes be used. Microorganism hosts are selected which are known to occupy the “phytosphere” (phylloplane, phyllosphere, rhizosphere, and/or rhizoplane) of one or more crops of interest. These microorganisms are selected so as to be capable of successfully competing in the particular environment (crop and other insect habitats) with the wild-type microorganisms, provide for stable maintenance and expression of the gene expressing the polypeptide pesticide, and, desirably, provide for improved protection of the pesticide from environmental degradation and inactivation.

A large number of microorganisms are known to inhabit the phylloplane (the surface of the plant leaves) and/or the rhizosphere (the soil surrounding plant roots) of a wide variety of important crops. These microorganisms include bacteria, algae, and fungi. Of particular interest are microorganisms, such as bacteria, e.g., genera Pseudomonas, Erwinia, Serratia, Klebsiella, Xanthomonas, Streptomyces, Rhizobium, Rhodopseudomonas, Methylophilius, Agrobactenum, Acetobacter, Lactobacillus, Arthrobacter, Azotobacter, Leuconostoc, and Alcaligenes; fungi, particularly yeast, e.g., genera Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobactenium tumefaciens, Rhodopseudomonas spheroides, Xanthomonas campestris, Rhizobium melioti, Alcaligenes entrophus, and Azotobacter vinlandii; and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces roseus, S. odorus, Kluyveromyces veronae, and Aureobasidium pollulans. Of particular interest are the pigmented microorganisms.

A wide variety of ways are available for introducing a B.t. gene encoding a toxin into a microorganism host under conditions which allow for stable maintenance and expression of the gene. These methods are well known to those skilled in the art and are described, for example, in U.S. Pat. No. 5,135,867, which is incorporated herein by reference.

Treatment of cells. Bacillus thuringiensis or recombinant cells expressing the B.t. toxins can be treated to prolong the toxin activity and stabilize the cell. The pesticide microcapsule that is formed comprises the B.t. toxin or toxins within a cellular structure that has been stabilized and will protect the toxin when the microcapsule is applied to the environment of the target pest. Suitable host cells may include either prokaryotes or eukaryotes, normally being limited to those cells which do not produce substances toxic to higher organisms, such as mammals. However, organisms which produce substances toxic to higher organisms could be used, where the toxic substances are unstable or the level of application sufficiently low as to avoid any possibility of toxicity to a mammalian host. As hosts, of particular interest will be the prokaryotes and the lower eukaryotes, such as fungi.

The cell will usually be intact and be substantially in the proliferative form when treated, rather than in a spore form, although in some instances spores may be employed.

Treatment of the microbial cell, e.g., a microbe containing the B.t. toxin gene or genes, can be by chemical or physical means, or by a combination of chemical and/or physical means, so long as the technique does not deleteriously affect the properties of the toxin, nor diminish the cellular capability of protecting the toxin. Examples of chemical reagents are halogenating agents, particularly halogens of atomic no. 17-80. More particularly, iodine can be used under mild conditions and for sufficient time to achieve the desired results. Other suitable techniques include treatment with aldehydes, such as glutaraldehyde; anti-infectives, such as zephiran chloride and cetylpyridinium chloride; alcohols, such as isopropyl and ethanol; various histologic fixatives, such as Lugol iodine, Bouin's fixative, various acids and Helly's fixative (See: Humason, Gretchen L., Animal Tissue Techniques, W. H. Freeman and Company, 1967); or a combination of physical (heat) and chemical agents that preserve and prolong the activity of the toxin produced in the cell when the cell is administered to the host environment. Examples of physical means are short wavelength radiation such as gamma-radiation and X-radiation, freezing, UV irradiation, lyophilization, and the like. Methods for treatment of microbial cells are disclosed in U.S. Pat. Nos. 4,695,455 and 4,695,462, which are incorporated herein by reference.

The cells generally will have enhanced structural stability which will enhance resistance to environmental conditions. Where the pesticide is in a proform, the method of cell treatment should be selected so as not to inhibit processing of the proform to the mature form of the pesticide by the target pest pathogen. For example, formaldehyde will crosslink proteins and could inhibit processing of the proform of a polypeptide pesticide. The method of treatment should retain at least a substantial portion of the bio-availability or bioactivity of the toxin.

Characteristics of particular interest in selecting a host cell for purposes of production include ease of introducing the B.t. gene or genes into the host, availability of expression systems, efficiency of expression, stability of the pesticide in the host, and the presence of auxiliary genetic capabilities. Characteristics of interest for use as a pesticide microcapsule include protective qualities for the pesticide, such as thick cell walls, pigmentation, and intracellular packaging or formation of inclusion bodies; survival in aqueous environments; lack of mammalian toxicity; attractiveness to pests for ingestion; ease of killing and fixing without damage to the toxin; and the like. Other considerations include ease of formulation and handling, economics, storage stability, and the like.

Growth of cells. The cellular host containing the B.t. insecticidal gene or genes may be grown in any convenient nutrient medium, where the DNA construct provides a selective advantage, providing for a selective medium so that substantially all or all of the cells retain the B.t. gene. These cells may then be harvested in accordance with conventional ways. Alternatively, the cells can be treated prior to harvesting.

The B.t. cells producing the toxins of the invention can be cultured using standard art media and fermentation techniques. Upon completion of the fermentation cycle the bacteria can be harvested by first separating the B.t. spores and crystals from the fermentation broth by means well known in the art. The recovered B.t. spores and crystals can be formulated into a wettable powder, liquid concentrate, granules or other formulations by the addition of surfactants, dispersants, inert carriers, and other components to facilitate handling and application for particular target pests. These formulations and application procedures are all well known in the art.

Formulations. Formulated bait granules containing an attractant and spores, crystals, and toxins of the B.t. isolates, or recombinant microbes comprising the genes obtainable from the B.t. isolates disclosed herein, can be applied to the soil. Formulated product can also be applied as a seed-coating or root treatment or total plant treatment at later stages of the crop cycle. Plant and soil treatments of B.t. cells may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other pesticidal additives, or surfactants. Liquid formulations may be aqueous-based or non-aqueous and employed as foams, gels, suspensions, emulsifiable concentrates, or the like. The ingredients may include rheological agents, surfactants, emulsifiers, dispersants, or polymers.

As would be appreciated by a person skilled in the art, the pesticidal concentration will vary widely depending upon the nature of the particular formulation, particularly whether it is a concentrate or to be used directly. The pesticide will be present in at least 1% by weight and may be 100% by weight. The dry formulations will have from about 1-95% by weight of the pesticide while the liquid formulations will generally be from about 1-60% by weight of the solids in the liquid phase. The formulations will generally have from about 10.sup.2 to about 10.sup.4 cells/mg. These formulations will be administered at about 50 mg (liquid or dry) to 1 kg or more per hectare.

The formulations can be applied to the environment of the lepidopteran pest, e.g., foliage or soil, by spraying, dusting, sprinkling, or the like.

Transfer (or introgression) of the Cry1F and Cry1Ab trait(s) into inbred maize lines can be achieved by recurrent selection breeding, for example by backcrossing. In this case, a desired recurrent parent is first crossed to a donor inbred (the non-recurrent parent) that carries the appropriate gene(s) for the Cry1F and Cry1Ab traits. The progeny of this cross is then mated back to the recurrent parent followed by selection in the resultant progeny for the desired trait(s) to be transferred from the non-recurrent parent. After three, preferably four, more preferably five or more generations of backcrosses with the recurrent parent with selection for the desired trait(s), the progeny will be heterozygous for loci controlling the trait(s) being transferred, but will be like the recurrent parent for most or almost all other genes (see, for example, Poehlman & Sleper (1995) Breeding Field Crops, 4th Ed., 172-175; Fehr (1987) Principles of Cultivar Development, Vol. 1: Theory and Technique, 360-376).

All patents, patent applications, provisional applications, and publications referred to or cited herein are incorporated by reference in their entirety to the extent they are not inconsistent with the explicit teachings of this specification.

The following examples illustrate the invention. The examples should not be construed as limiting.

### EXAMPLE 1

An O. nubilalis population that was approximately 1200× resistant to Cry1F was created via a laboratory selection program. Larvae from this Cry1F-resistant population and the corresponding non-selected (i.e., Cry1F sensitive) population were assessed for their ability to survive on plant tissue from near-isogenic corn genotypes expressing Cry1F, Cry1Ab or no insecticidal protein (i.e., non-transgenic).

Bioassays were conducted on sections of leaves from each corn genotype using neonate O. nubilalis. Leaf sections approximately 2 cm² in area were placed in each well of a 32 well plastic tray containing solidified agar/water. Each leaf section was infested with 50-60 neonate O. nubilalis. The trays were sealed with a ventilated mylar lid and held at 26° C. for 3days. After 3 days, larval feeding on the leaf sections was assessed using the rating system summarized in Table 1. Leaves from approximately 50 plants of each genotype were bioassayed in this experiment.

TABLE 1
Rating system for assessing larval feeding damage on corn leaf sections.

| Rating | Description |
|---|---|
| 1 | No feeding to a few pin holes |
| 2 | Several pin holes, no lesions |
| 3 | Pin holes common plus a few small lesions no larger than a pin |
| | head |
| 4 | Small lesions common |
| 5 | Large, elongated lesions larger than a pin head to completely |
| | destroyed |

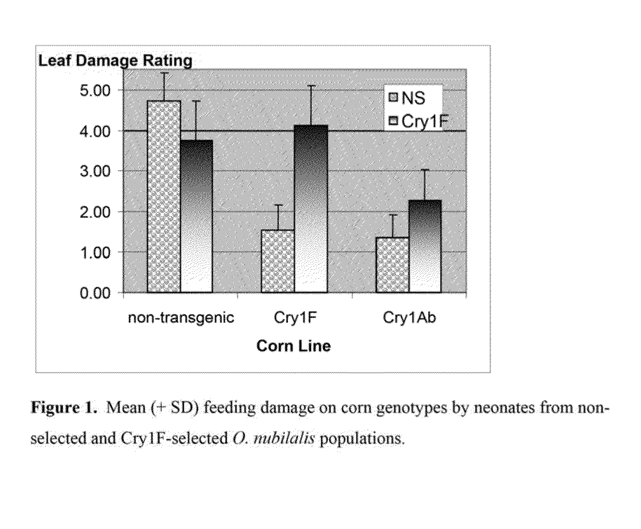

The results from this experiment are summarized in FIG. 1. O. nubilalis larvae from the Cry1F-selected and non-selected populations consumed approximately equal quantities of non-transgenic leaf tissue. Larvae from the Cry1F-selected population consumed significantly more Cry1F-expressing leaf tissue than larvae from the non-selected population. O. nubilalis larvae from the Cry1F-selected and non-selected populations consumed approximately equal quantities of Cry1Ab-expressing leaf tissue.

### EXAMPLE 2

The O. nubilalis population of Example 1 was further characterized in laboratory studies. These studies were designed to (1) quantify the level of resistance to Cry1Fa relative to a susceptible laboratory population, (2) identify potential cross-resistance to Cry1Ab, and (3) determine the genetic basis of resistance (i.e., monogenic vs. polygenic, autosomal vs. sex-linked).

The Cry1Fa-selected colony described in Example 1 was maintained by exposing neonate larvae to a concentration of Cry1Fa that corresponded to the upper limit of the 95% confidence interval of the LC₉₉ derived from assessments of Cry1Fa-susceptible field populations. Individual neonate larvae (at least 1,000 per generation) were exposed to artificial diet in which the diet surface was treated with Cry1Fa. Surviving larvae (those that had initiated feeding and grown beyond first instar) were transferred to untreated diet and reared to adults using standard rearing techniques. A Cry1Fa susceptible colony was established from the same starting population by taking individuals exposed to the diagnostic concentration that had not grown beyond the first instar but were still alive and transferring them to untreated diet. These larvae were reared to adults using standard rearing techniques.

Bioassays of neonate larvae were conducted to quantify the sensitivity of the Cry1Fa-selected and susceptible populations to Cry1Fa and Cry1Ab. These bioassays used the techniques previously developed for bioassays of Cry1Ab (Marçon et al. 1999). Briefly, dilutions of Cry1Fa and Cry1Ab were prepared in water containing 0.1% Triton-X 100 and were applied to the surface of artificial diet in individual wells of a 128 well trays (each well 16 mm diam.×16 mm height; CD International, Pitman, N.J.). After the solution dried, an individual neonate larva (less than 24 h after hatching) was placed in each well. Mortality and the pooled weight of surviving larvae from each treatment were recorded after 7 days. The control treatment consisted of wells treated with water containing 0.1% Triton-X 100 only. Bioassays were repeated on two different dates and included at least five Bt concentrations producing mortality that was greater than 0% but less than 100%. Mortality in the Bt treatments was corrected for control mortality and lethal concentrations with 95% fiducial limits were calculated using probit analysis (Finney 1971, LeOra Software 1987; Robertson & Preisler 1992). Larval weights were transformed to % growth inhibition relative to the controls and these data were analyzed by non-linear regression (SAS Institute Inc. 1988).

The inheritance of resistance in the Cry1Fa-selected population was determined using reciprocal crosses of resistant and susceptible parents. The F1 progeny from reciprocal crosses were bioassayed for Cry1Fa susceptibility using techniques described above. The mortality curves were evaluated for sex-linkage and for the degree of dominance (Stone 1968).

Results from concentration-response bioassays on the Cry1Fa-selected and susceptible populations are presented in Tables 2 and 3. The level of resistance in the Cry1Fa-selected population was so high that it was not possible, within the limits of the bioassay method, to produce mortality over the range of concentrations tested. Therefore, it was not possible to calculate the LC₅₀ (Table 1), and the resistance ratio (LC₅₀ resistant population/LC₅₀ susceptible population) is estimated to be much greater than the ratio of the highest Cry1Fa concentration tested to the LC₅₀ of the susceptible population ([12,000 ng/cm²]/[3.6 ng/cm²]=3333). The results for growth inhibition were similar to those for mortality in that the response of the Cry1Fa-selected population to Cry1Fa was insufficient to allow for an estimate of the EC₅₀ (Table 3). The resistance ratio is estimated to be much greater than the ratio of the highest Cry1Fa concentration tested to the EC₅₀ of the susceptible population ([12,000 ng/cm²]/[0.54 ng/cm²]=22,222). The Cry1Fa-selected and susceptible populations were equally susceptible to Cry1Ab (Tables 2 and 3).

TABLE 2
Sensitivity of Cry1Fa-selected and susceptible European corn borer populations
to Cry1Fa and Cry1Ab based on larval mortality. LC values are expressed as ng/cm².

| Toxin | Colony | N | Slope ± SE¹ | LC₅₀ (95% FL) | LC₉₀ (95% FL) | LC₉₉ (95% FL) | χ² | RR² |
|---|---|---|---|---|---|---|---|---|
| Cry1Ab | Control | 445 | 3.09 ± 0.39 | 1.23 (0.89-1.59) | 3.21 (2.33-6.17) | 6.99 (4.20-22.66) | 8.2 | — |
| | Selected | 446 | 2.20 ± 0.22 | 1.84 (1.44-2.46) | 7.05 (4.65-14.04) | 21.06 (11.21-62.50) | 5.75 | 1.5 |
| Cry1Fa | Control | 448 | 1.69 ± 0.16 | 3.60 (2.42-4.98) | 20.74 (13.89-37.94) | 86.43 (45.33-252.81) | 6.45 | — |
| | Selected | 448 | | >>12000 | | | | >3333 |

¹Data were analyzed by probit analysis using Polo-PC (LeOra Software 1987).
²Resistance Ratio = selected colony (R) LC₅₀/control colony (S) LC₅₀.

TABLE 3
Sensitivity of Cry1Fa-selected and susceptible European corn borer populations
to Cry1Fa and Cry1Ab based on growth inhibition. EC values are expressed as ng/cm².

| Toxin | Colony | N | EC₅₀ (95% FL)¹ | EC₉₀ (95% FL) | EC₉₉ (95% FL) | RR² |
|---|---|---|---|---|---|---|
| Cry1Ab | Control | 445 | 0.17 (0.13-0.21) | 0.98 (0.73-1.46) | 6.69 (3.83-14.93) | — |
| | Selected | 446 | 0.23 (0.18-0.28) | 1.35 (1.00-2.02) | 9.42 (5.36-21.02) | 1.35 |
| Cry1Fa | Control | 448 | 0.54 (0.48-0.59) | 2.15 (1.78-2.62) | 9.84 (6.03-14.02) | — |
| | Selected | 448 | >>12000 | | | >22222 |

¹Larval weights were transformed to percent growth inhibition relative to controls and analyzed by logit analysis (Robertson & Preisler 1992).
²Resistance Ratio = selected colony (R) EC₅₀/control colony (S) EC₅₀.

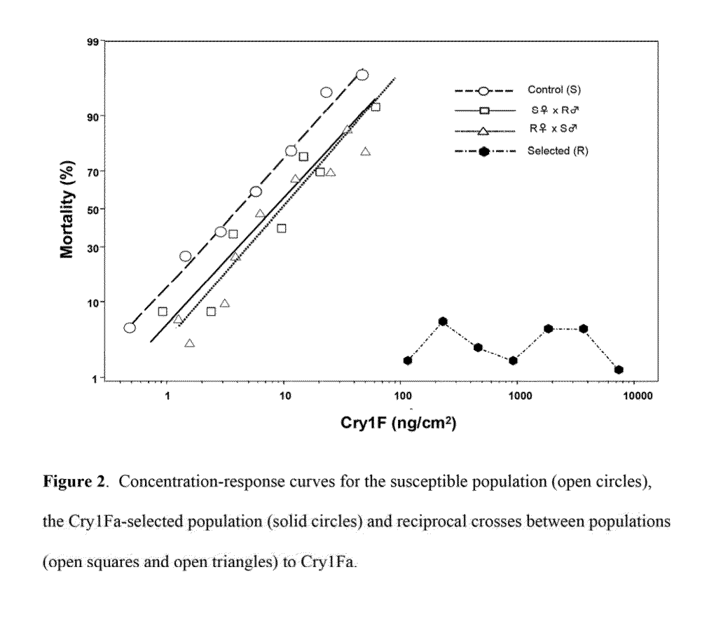

Results of reciprocal crosses of the Cry1Fa-selected and susceptible parental populations are presented in FIG. 2. These data provide no evidence of sex-linked heritability as the concentration-mortality curves for both individual reciprocal crosses are not significantly different (P<0.05). In addition, Cry1Fa resistance appears to be recessive as the progeny dose-response curves are much more similar to the susceptible parent population than the Cry1Fa-resistant parent population.

In summary, these data document a high level of resistance to Cry1Fa in an O. nubilalis population that was selected for resistance in the laboratory. The recessive nature of this resistance supports the utility of the high-dose/refuge resistance management strategy employed for transgenic corn expressing Cry1Fa. The fact that this Cry1Fa-selected population is fully susceptible to Cry1Ab is strong evidence to the value of deploying a stack of Cry1Fa and Cry1Ab as a resistance management strategy.

### REFERENCES

Finney, D. J. 1971. Probit analysis. Cambridge University Press, England.
Hua, G., L. Masson, J. L. Jurat-Fuentes, G. Schwab, and M. J. Adang. Binding analyses of Bacillus thuringiensis Cry d-endotoxins using brush border membrane vesicles of Ostrinia nubilalis. Applied and Environmental Microbiology 67[2], 872-879. 2001.
LeOra Software. 1987. POLO-PC. A user's guide to probit and logit analysis. Berkeley, Calif.
McGaughey, W. H., F. Gould, and W. Gelernter. Bt resistance management. Nature Biotechnology 16[2], 144-146. 1998
Marçon, P. R. G. C., L. J. Young, K. Steffey, and B. D. Siegfried. 1999. Baseline susceptibility of the European corn borer, Ostrinia nubilalis (Hübner) (Lepidoptera: Pyralidae) to Bacillus thuringiensis toxins. J. Econ. Entomol. 92 (2): 280-285.
Robertson, L. J. and H. K. Preisler. 1992. Pesticide bioassays with arthropods. CRC Press, Boca Ranton, Fla.
SAS Institute Inc. 1988. SAS procedures guide, Release 6.03 edition. SAS Institute Inc, Cary, N.C.
Stone, B. F. 1968. A formula for determining degree of dominance in cases of monofactorial inheritance of resistance to chemicals. Bull. WHO 38:325-329.
Van Mellaert, H., J. Botterman, J. Van Rie, and H. Joos. Transgenic plants for the prevention of development of insects resistant to Bacillus thuringiensis toxins. (Plant Genetic Systems N.V., Belg. 89-401499[400246], 57-19901205. EP. 5-31-1989

1. A method of controlling, or preventing the development of a population of, Cry1F-resisant European corn borers, said method comprising deploying transgenic corn plants in combination with a refuge, wherein said transgenic corn plants produce a Cry1Fa protein and a Cry1Ab protein.

2. A composition for controlling European corn borers, said composition comprising cells that express effective amounts of both a Cry1Fa chimeric core toxin-containing protein and a Cry1Ab chimeric core toxin-containing protein.

3. The composition of claim 2 comprising a host transformed to express both a Cry1F core toxin-containing protein and a Cry1Ab core toxin containing protein, wherein said host is a corn plant cell.

4. A method of controlling European corn borers, said method comprising presenting to said pests or to the environment of said pests an effective amount of the composition of claim 2.

5. A maize plant comprising DNA encoding a Cry1Ab chimeric core toxin-containing protein and DNA encoding a Cry1F core toxin-containing protein.

6. Seed of the plant of claim 5, said seed comprising DNA encoding a Cry1Ab chimeric core toxin-containing protein and DNA encoding Cry1F core toxin-containing protein.

7. A method of controlling Cry1F-resistant European corn borers, said method comprising presenting to said pests or to the environment of said pests an effective amount of a Cry1Ab protein.
